(19) United States
(12) Patent Application Publication
(10) Pub. No.: US 2022/0132954 A1
(43) Pub. Date: May 5, 2022

(54) A Hygienic Water-Soluble Glove

(71) Applicant: Soluglove, LLC, Philadelphia, PA (US)

(72) Inventor: Agustin Legido, Philadelphia, PA (US)

(73) Assignee: Soluglove, LLC, Philadelphia, PA (US)

(21) Appl. No.: 17/431,794

(22) PCT Filed: Feb. 17, 2020
(86) PCT No.: PCT/US20/18481

§ 371 Date: Aug. 18, 2021

Related U.S. Application Data
(60) Provisional application No. 62/806,929, filed on Feb. 18, 2019.

(51) Int. Cl. A41D 19/00; A47K 7/08; A47K 7/04

(52) U.S. Cl. CPC A41D 19/0075; A47K 7/043; A47K 7/08; A41D 19/0082

(57) ABSTRACT

Systems, methods and apparatus are provided for a water soluble hygienic glove. The glove includes a first layer, a second layer, an opening and a sealed perimeter. The opening is adapted to accommodate insertion of a user's hand into the opening and into a formed cavity. All possible shapes of gloves are considered. One embodiment has an outer perimeter designed for dispensing gloves in a role where each glove has an outer perimeter in a rectangular form and joined through a line of perforations which can be easily torn to separate a glove. The user easily inserts a hand in the glove for subsequent cleaning of the rectal area or other areas of an individual and then disposes of the used glove in a flush toilet where it readily dissolves in the presence of water. The glove is convenient and safe for conventional septic systems and/or sewage treatment facilities. The present invention further considers storage and dispensing devices. Other embodiments incorporate the use of germicidal agents and drying agents. The system is applicable in commercial restrooms or bathrooms in private homes.

### CROSS REFERENCE TO RELATED APPLICATIONS

This application is the US national application of PCT/US2020/018481, filed 17 Feb. 2020, and which claims benefit of and priority to U.S. Provisional Patent Application No. 62/806,929, filed 18 Feb. 2019, now expired, the disclosure of which is incorporated by reference in its entirety.

### FIELD OF THE INVENTION

The invention relates to systems, methods and apparatus for optimizing personal care and providing hygienic protection during toilet stops. More specifically, a water soluble hygienic glove is disclosed for use in sanitation as a personal care adjunct, medical, health care, and in veterinary personal pet care.

### BACKGROUND OF THE INVENTION

This invention described herein relates generally to a means for hygienic hand protection generally used in rectal care, cleaning skin surfaces, and/or handling unclean items. The invention is useful when cleaning any surface, especially on a regular cleaning basis where disposal through a flush toilet is readily available. This may include surfaces found in bathrooms, kitchens, nursing homes, emergency rooms, and hospital patient rooms. A hygienic glove is most applicable when cleaning skin or bodily orifices. Generally, any unclean items that may need to be handled which may include, for example, feces, soiled clothing, bloody items, used sanitary napkins, used tampons, or used diapers. A hygienic glove is especially useful in securing toilet paper to wipe an individual's bodily orifices and, generally, for cleaning an unsanitary surface. The invention goes well beyond the application of toilet paper or toilet paper plus individual wipes post defecation. Specifically, this invention enables the user to more thoroughly clean the target surface, resulting in not only a cleaner rectal area post defecation but also superior hand sanitation, decreased toilet paper use, and reduced undergarment soiling, together with the added benefit of quick and easy disposal through the flush toilet.

Prior to the disclosure of the present invention, one option would be for a person to wear form-fitting gloves made of rubber or latex and use a paper towel, a length of toilet paper, a baby wipe, a moist towelette, a hand towel, or a sponge, to wipe the unclean surface or handle the unclean item. Then dispose of the glove in a solid waste container. This can be very tedious and impractical in many routine circumstances and does not remove the presence of any odor originating in the removed waste. One alternative, such as at home and not involving particularly dangerous substances or diseases, users may just forego using a latex or rubber glove and not have any protection. For instance, a person changing a baby's diaper may choose to only use baby wipes to wipe away feces on the baby's skin and anus, without the use of gloves. Instead of wearing gloves, a user may rely on hand-washing after changing a baby's diaper. The same is true by an individual immediately post defecation where an individual will take a predetermined length of toilet paper with their dominant hand followed by a wiping motion of the rectal area. In these situations, the soiled paper is immediately dropped into a flush toilet water for disposal and the user will presumably wash their hands. When only relying on hand-washing there is always the risk that the hands are not completely clean, thus increasing the risk of contaminating the user and other contact surfaces and objects which is especially important in the food industry.

In situations where reliance is solely on hand-washing after defecation, a major disadvantage is that there will be no personal protection, increasing the risk of contamination. Toilet paper may rip if it gets wet or moist, increasing the chances of contact. Because toilet paper usually is found near a flush toilet and may be flushed, toilet paper is a convenient choice for individuals or caregivers in wiping a backside, but the convenience factor decreases greatly if the toilet paper falls apart. Likewise, a caregiver of an adult patient may not be in a position to see an area being wiped if the patient is sitting on a flush toilet. As such, a caregiver may opt for a non-flushable towelette or wipe, to avoid the toilet paper coming apart while wiping, but the consequence is that a feces-laden wipe is placed in a garbage container, where it may emit foul odors and potentially leave a trail of fecal matter or inappropriately flushed down a flush toilet.

A still further problem with continued glove and wipe use, especially in densely populated areas, is the excessive buildup of trash. Large apartment buildings and metropolitan areas have established policies and rules to address the problem, but these regulations are difficult to enforce and do not resolve the trash buildup problem.

More globally, a problem in many over populated urban cities is the formation of a congealed mass formed within sewer systems by the combination of flushed non-biodegradable solid matter, such as wet wipes and congealed grease or cooking fat or Fatbergs which became a problem in the 2010s in Britain, because of ageing Victorian sewers and the rise in usage of disposable (so-called “flushable”) cloths.

Currently, one alternative is to clean surfaces or handle unclean items while wearing a disposable glove, usually capable of being decomposed by bacteria or other living organisms, which is removed after use and placed into the garbage for decomposition in a landfill or compost. These disposable gloves are intended for single use and not intended to be re-used, or to be worn, removed, and then worn again. The problem, in part, includes proper disposal of these one-use disposable gloves after use, especially when used to clean an individual post defecation. Again, there are the issues of placing the glove in a solid waste disposal container and the subsequent release of odors.

Further, there are disposal problems involving the lack of sufficient bacterial decomposition of the material used in sanitary protection. Current disposable gloves on the market are decomposed through composting, but again the immediate disposal of these gloves can become a problem if placed in a garbage container after use.

Further, disposal problems associated with pet care require safe environmental techniques that reduce the buildup of animal waste and compose. Pets owners living in Urban areas and having dogs need to clean up after their dog during frequent walks, especially in the same urban outdoors such as parks or places that require owners to clean up after their pets. Previous methods included such protection as a special removable gloves, portable containers or other means used by the owners to collect the dog's waste and deposit the isolated waste in the garbage or other storage containers.

In light of the disadvantages of conventional disposable gloves using wipes, sheets, towelettes, paper towels, and toilet paper lengths, a new type of glove having an alternate disposal means is desirable that allows for more efficient and/or effective use of the disposable barrier and protection of the user's hand. Additionally, the improved means should allow for sanitary disposal at the point of use.

A need exists for an easily accessible protection means to completely and effectively protect the user as they are wiping a rectal surface after defecation and then provide for the efficient and environmentally safe disposal of the protection means.

### SUMMARY OF THE INVENTION

The present invention provides, in part, for a water-soluble glove for use in cleaning the rectal area of an individual or other areas of an individual and then disposing of the used glove in a flush toilet. The glove is incorporated into systems, methods and apparatus where the use of the glove provides for a hygienic means for water soluble disposal of the glove. The glove has a first layer and second layer, an opening and a sealed perimeter. The opening is adapted to accommodate insertion of a user's hand into the opening and into a formed cavity within the sealed perimeter to include fingers and the palm area. All possible shapes of gloves are considered including, but not limited to, a rectangle, a thumbed-mitten, a five-fingered glove, or a combination of these. One embodiment has an outer perimeter designed for dispensing gloves in a roll where each glove has an outer perimeter in a rectangular form and joined through a line of perforations which can be easily torn to separate a glove. The user easily inserts a hand in the glove for subsequent cleaning of the rectal area or other areas of an individual and then disposes of the used glove in a flush toilet where it readily dissolves in the presence of water. The glove is convenient and safe for conventional septic systems and/or sewage treatment facilities. The present invention further considers storage and dispensing devices. Other embodiments incorporate the use of germicidal agents and drying agents. The system is applicable in commercial restrooms or bathrooms in private homes

While all known means for the manufacture and formation of gloves is considered, one embodiment of the invention incorporates a water soluble hygienic glove, formed in a two-layer process. The glove includes a first layer, a second layer, an opening and a sealed perimeter. The opening is adapted to accommodate insertion of a user's hand into the opening and into a cavity between, and formed by, the first layer and the second layer. The first layer has a first perimeter edge, the second layer has a second perimeter edge, and the first perimeter edge and the second perimeter edge are joined at the sealed perimeter and are not joined at the opening. The sealed perimeter and the opening form a total perimeter of the glove. The glove is designed for use as either a left-fitting or right-fitting glove. Embodiments include, but not limited to, a rectangle, a thumbed-mitten, a five-fingered glove, or a combination of these.

The glove comprises a disposable material that is soluble in water and is safe for disposal in a flush toilet emptying into conventional septic systems and/or sewage treatment facilities. The water-soluble glove is comprised of the following components: a polyvinyl alcohol; a composite plasticizer; a polysaccharide derivative; a processing aid; a film slipping agent; and a film anti-blocking agent (WO2018041262).

Once the glove is placed into the water in the toilet, the material rapidly dissolves into solution forming a non-toxic liquid. The dissolved material and water are easily removed by flushing the toilet.

In another embodiment of the invention, a device is provided that includes a hygienic glove dispenser containing a roll of multiple disposable, water-soluble hygienic gloves. The gloves are contained in a glove dispenser in a dispensing form that makes individual glove removal easy and ready for use. The dispensing form may comprise the water-soluble gloves in a form where individual gloves are attached through a line of perforations. Tearing the perforations will separate a new water-soluble glove ready for use, much like a standard toilet paper dispenser.

Alternatively, multiple water-soluble gloves may be stacked within the dispenser, similar to a tissue box dispensing facial tissues. In one embodiment, a water-soluble glove is taken from overlapping folds in the stack for use.

In another embodiment, a pair of water-soluble gloves are sealed in a small, air-tight package for single use. The user is able to carry the small package in a personal hand bag so that, for example, when needed in a public restroom the user simply opens the package to access the water-soluble gloves. This is very useful in changing a woman's tampon or other feminine hygiene device.

A still further embodiment of the present invention Is the use of a water-soluble glove, either as a pair, or more preferred as a single glove suitable for quickly clutching a pets waste matter after discharged from the animals bowels or feces. Once the pet owner is able to clutch the feces, they easily slip the remaining outer portion of the glove over the fecal material and deposit the combined glove-fecal material into a garbage container or other land file to allow the glove to dissolve and captured feces to decompose.

Other features and aspects of this invention will become more fully apparent from the following detailed description, the appended claims and the accompanying drawings.

### BRIEF DESCRIPTION OF THE DRAWINGS

FIGS. 1A-B show perspective views of exemplary components of a dissolvable glove.

FIGS. 2A-C show perspective views of exemplary embodiments of selected shapes for the glove.

FIG. 3 shows a perspective view of a combination of a rectangular five-fingered glove.

FIG. 4 shows a glove roll dispenser for a rectangular shaped glove.

### DESCRIPTION OF THE PREFERRED EMBODIMENTS

The figures and descriptions of the present invention are presented in a simplified format to illustrate elements that are relevant for a clear understanding of the present invention. Those of ordinary skill in the art will recognize that other elements may be desirable and/or required in order to implement the present invention. Because such elements are well known in the art and because they do not facilitate a better understanding of the present invention, a discussion of such elements is not provided herein. It should also to be understood that the drawings included herewith only provide diagrammatic representations of the presently preferred structures of the invention and that other structures falling within the scope of the present invention may include structures different than those shown in the drawings.

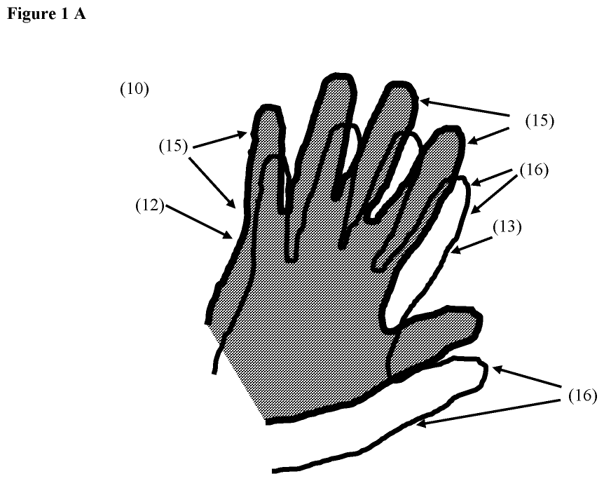

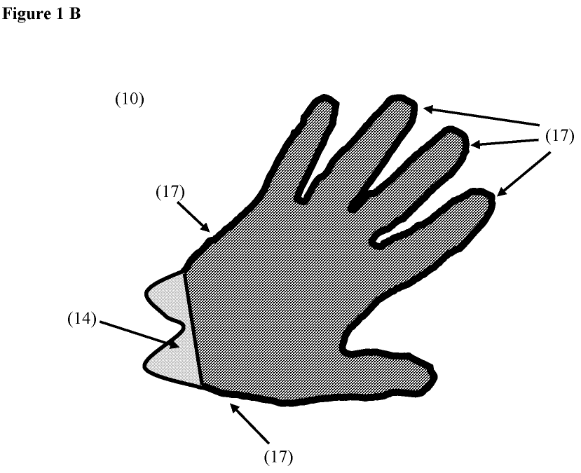

Referring to FIGS. 1A-B, the invention relates to a water-soluble glove, such as for cleaning, personal sanitation and/or hygiene purposes. The glove (10) includes a first layer (12), a second layer (13), an opening (14) and a sealed perimeter (17). The opening is adapted to accommodate insertion of a user's hand into the opening and into a cavity between, and formed by, the first layer and the second layer. The first layer (12) has a first perimeter edge (15), the second layer (13) has a second perimeter edge (16), and the first perimeter edge (15) and the second perimeter edge (16) are joined as the sealed perimeter (17) and are not joined at the opening (14). The sealed perimeter and the opening form a total perimeter of the glove (FIG. 1B). The glove is designed for use as a left-fitting or right-fitting glove.

Exemplary embodiments may include, but not limited to, a rectangle, a thumbed-mitten, a five-fingered glove, or a combination of these. For example, a rectangular first layer and a rectangular second layer may be sealed to form a rectangular thumbed-mitten or a rectangular five-fingered glove. As shown in FIG. 1B, one embodiment of the invention may include a rectangular five-fingered glove. Other dimensions are conceivable also and depend on the means for dispensing. The opening width (14) refers to how wide the opening is into which a user inserts a hand into a cavity formed by joining the first layer and the second layer.

The glove comprises a disposable material that is soluble in water and is safe for disposal in a flush toilet emptying into conventional septic systems and/or sewage treatment facilities. The water-soluble glove is comprised of the following components: a polyvinyl alcohol; a composite plasticizer; a polysaccharide derivative; a processing aid; a film slipping agent; and a film anti-blocking agent. More preferably, the water-soluble glove comprises the following components according to their individual parts by weight: 100 parts of polyvinyl alcohol; 8-20 parts of composite plasticizer; 10-30 parts of a polysaccharide derivative; 1-5 parts of a processing aid; 0.1-0.5 parts of a film slipping agent; and 0.5-3.0 parts of a film anti-blocking agent (WO2018041262).

The preparation method comprises the following steps: mixing the components according to the parts by weight, extruding the mixture by means of a twin screw extruder to form pellets after mixing, then preforming blow molding to form a film by means of a thermoplastic extrusion and film blowing machine, and then preparing various types and sizes of water-soluble gloves by means of a glove machine. The water-soluble glove has higher bearing capacity and cold-water solubility, making it suitable for use in water-free environment, and after use dissolves upon contacting water without producing pollution.

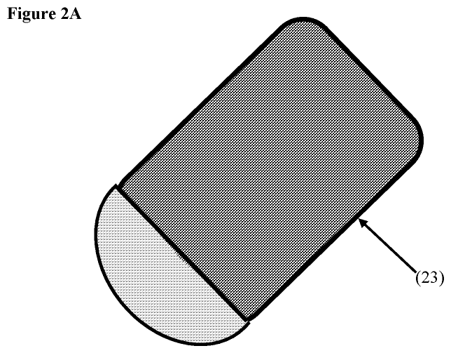

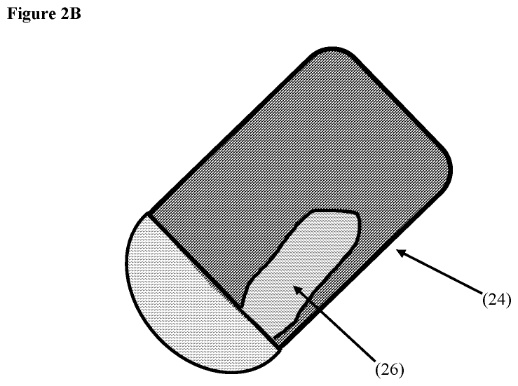

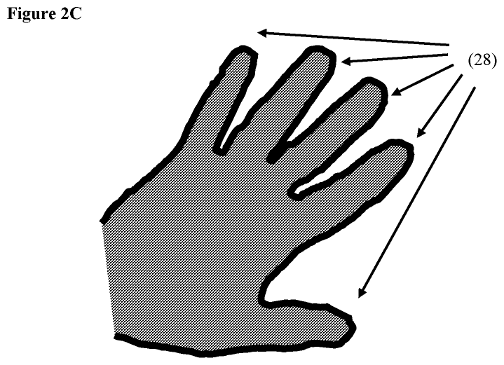

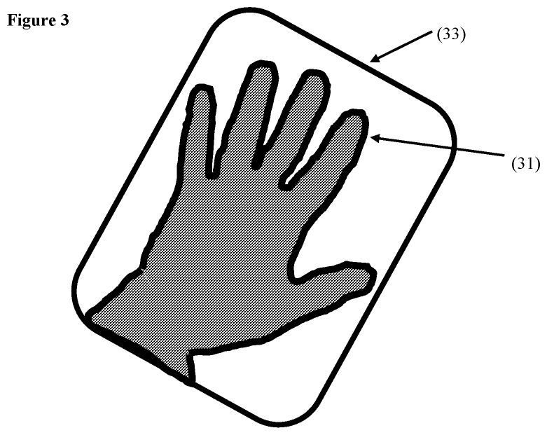

Exemplary embodiments of the different shapes are shown in FIG. 2A-C and may include, but not limited to, a rectangle (FIG. 2A) having an outer rectangular perimeter (23), a thumbed-mitten (FIG. 2B) having an inner area for a thumb (24) and an outer rectangular perimeter (26), a five-fingered glove (FIG. 2C) having individually separate finger placements (28), or a combination of these. For example, a rectangular first layer and a rectangular second layer may be sealed to form a rectangular thumbed-mitten or a rectangular five-fingered glove (FIG. 3). The general rectangular form may provide better performance for the glove when dispensed on a continuous dispensing roll.

Optionally, each water-soluble glove may be coated on the outer surface with germicide or disinfectant agents to further reduce the risk of contamination. The germicides provide an additional mode of protection for the user.

Optionally, each water-soluble glove may be coated on the inside with a germicide or disinfectant agent that is not harmful to the user. Treating the inner portion of the glove with cornstarch or other powder allows for a more comfortable fit. One example, is the use of thymol in a dusting powder. Thymol is a naturally found product used for treating infections. It has been used as a safe and effective antiseptic ingredient in mouth wash, prevents mold and as a general multipurpose disinfectant.

Each embodiment of the present invention is designed to protect the user's hand from coming into direct contact with contaminants, such as fecal material, being handled or cleaned by a user. The fact that the user is less concerned about direct skin contact while cleaning the surface enables more cleaning power and efficiency in completing the procedure. The user will be able to cover a larger, potentially problematic surface area than under conventional non-gloved methods and will have less of a concern that the toilet paper will break up during use, thus providing a more efficient method of cleaning. After use, the water-soluble glove and toilet paper are disposed by pulling the glove off of the hand and putting in both in the toilet water and flushing in a flush toilet.

As shown in FIG. 3, the water-soluble glove may be a combination of a finger glove (31) and a rectangular outer “mitten” shape (33) where the rectangular shape provides for one to five individual finger insertions in an orientation similar to an inner five-finger glove, each finger inserts joined through a single seam. Alternatively, single or multiple individual finger inserts can be created within the rectangular shape. For example, individual finger inserts may be formed for the thumb and index finger. Having joined finger inserts may provide more control to a user while preventing waste or torn toilet paper from passing between the fingers. Alternatively, a glove having separated five-finger inserts may provide more dexterity to a user's hand to grip and handle the paper.

With a glove mitten or combination having an outer rectangular shape, an adult patient having less dexterity and mobility would be able to reach all the areas needed to clean after a bowel movement, or it may make a caregiver's task of following up with the adult patient more efficient, while providing more protection to the caregiver's hand. Moreover, a soluble mitten could be used with the application of a skin cream or alcohol-base lotion on a patient's back and bottom area and then easily disposed by putting the used glove in a flush toilet.

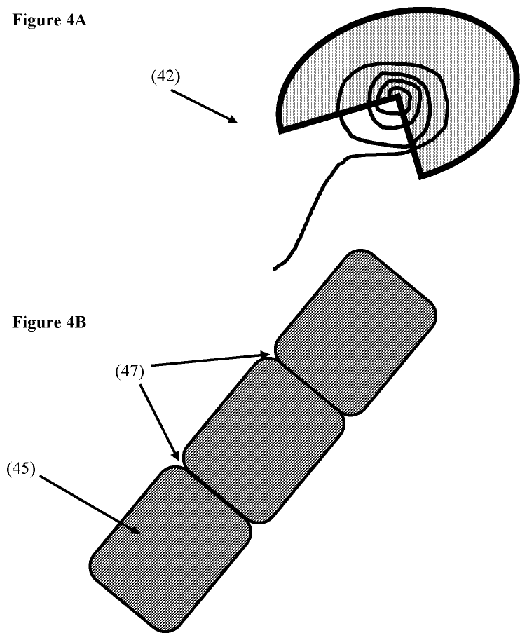

Referring to FIG. 4, water-soluble gloves may be dispensed from a modification of a toilet roll dispenser (42). For example, FIG. 4A depicts a covered dispenser commonly used in public restrooms. The gloves are designed with a combination rectangular outer surface having multi-fingered inserts (45), ranging from 1 to 5 fingered inserts. When the user pulls on the end glove, a unit of length from the roll is dispensed to allow the user to tear along a perforation (47) and separate the glove from the rest of the roll (FIG. 4B). The user then puts his hand in the glove and continues with removing toilet paper. With the toilet paper in the gloved hand, the user is able to easily and efficiently clean the rectal area. When finished, the user simply put the used paper and glove into a flush toilet and flushes.

Alternatively, the gloves may be individually stacked in a dispensing box modified from a facial tissue dispenser. Here, the user simply pulls out an individual glove, ready for use.

Alternatively, a pair of gloves may be packaged in sealed container for individual use. The packaged gloves are discretely stored in a small package that can be easily torn or opened prior to cleaning the rectal area. When opened, the user inserts a hand into the glove and begins the process of obtaining toilet paper and cleaning the rectal area. Once the cleaning is complete, the user simply places the gloves and toilet paper into a flush toilet and flushes.

A still further embodiment of the present invention is a water-soluble glove as described herein, either as a pair, or more preferred as a single glove. The glove is contained in a portable storage or storage dispensing device to allow the user to easily put their hand through while walking a pet with the expectation the pet will begin defecation, making use suitable for quickly clutching a pets waste matter after being discharged from the animals bowels or feces. Once the pet owner is able to clutch the feces, they easily slip the remaining outer portion of the glove over the fecal material and deposit the combined glove-fecal material into a garbage container or other land fill to allow the glove to dissolve in a moisture environment, allowing the captured feces to compose. The water-soluble glove is designed to quickly capture and remove animal feces. The design can be any reasonable shaped form to discretely remove or lift the feces. Other considerations for the outer shaped design are a mitten shaped glove, a flared rectangular shaped form or a five-finger shaped design. The glove is designed for use as either a left-fitting or right-fitting glove. Embodiments include these flaring designs and other but not limited to, a rectangle, a thumbed-mitten, a five-fingered glove, or a combination of these

Optionally each dispensing embodiment may comprise a desiccant. A desiccant can be any hydroscopic substance that induces or sustains a state of dryness within a local vicinity. Individually package glove pairs may further contain pre-packaged desiccants to extend shelf life and improve function the dry glove prior to use. Inert desiccants such as, but not limited to, silica is considered in the present invention. Other common desiccants include activated charcoal, calcium sulfate, calcium chloride and zeolites. Desiccants are especially appropriate in larger, roll dispensers where the glove roll may be exposed to long term humidity or moister in the air. A desiccant provides storage stability for the gloves.

Optionally in each dispensing embodiment a means for assessing the immediate moisture content around the glove provides information on the gloves integrity for use. Any humidity indicator know in the art is considered. Whether this indicator is included in the desiccant to show color changes or as a separate indicator, the humidity indicator provides an index of the glove status prior to use. A glove stored in packaging having a higher than usual moisture content would be vulnerable to instability and breakdown. An indicator provides a means to assess this instability prior to use. One common indicator is cobalt chloride. Anhydrous cobalt chloride is blue. When it bonds with two water molecules it turns purple. Further hydration results in pink-colored hexaaquacobalt(II) chloride complexes. Thus, by simply looking at the indicator color, a user will be able to rapidly determine the status of the glove and if the glove will keep its integrity during use.

The foregoing description discloses exemplary embodiments of the invention, which has been described with the reference to the exemplary embodiments. The specification and drawings, accordingly, should be regarded in an illustrative rather than restrictive sense. Various modifications and changes may be made to the embodiments without departing from the broadest spirit and scope of the invention. Modifications of the above disclosed apparatus and methods that fall within the scope of the invention will be readily apparent to those of ordinary skill in the art. Accordingly, additional embodiments may fall within the spirit and scope of the invention, as defined by the following claims.

Those of ordinary skill in the art will recognize that various modification and variation may be made to the embodiments described above without departing from the spirit and scope of the present invention.

I claim:

1. A disposable glove comprising:
a. a water-soluble material; and
b. a shape for hygienic wiping of a surface.

2. The glove of claim 1 having a water-soluble material comprising a polyvinyl alcohol.

3. The glove of claim 1 having a shape selected from the group consisting of a rectangle glove, a thumbed-mitten glove, a fingered glove and combination thereof.

4. The glove of claim 3 wherein the fingered glove has 1 to 5 fingers.

5. The glove of claim 4 wherein the fingered glove has a combination outer rectangular perimeter and inner fingered inserts.

6. The glove of claim 1 having a further inner or outer coating wherein the outer coating is a disinfectant.

7. The glove of claim 6 where the disinfectant is thymol.

8. The glove of claim 1 having an inner coating wherein the inner coating is a powder to provide the use with a comfortable fit.

9. The glove of claim 8 where the powder is cornstarch.

10. The glove of claim 1 further having a shape designed for capturing pet feces.

11. A hygienic system for clean the rectal area of an individual comprising:
a. a water-soluble glove;
b. a water-soluble glove dispenser;
c. toilet paper; and
d. a flush toilet.

12. The hygienic system of claim 10 having a water-soluble glove with a combination outer rectangular perimeter and inner fingered insert.

13. The hygienic system of claim 10 wherein the water-soluble glove dispenser is a roll of gloves individually attached through a perforated line.

14. The hygienic system of claim 10 wherein the water-soluble glove dispenser is a dispensing box with individual water-soluble gloves stacked inside.

15. The hygienic system of claim 10 wherein the water-soluble glove dispenser is a pre-packaged container for a single pair of water-soluble gloves.

16. The hygienic system of claim 10 wherein the water-soluble glove dispenser contains a desiccant.

17. The hygienic system of claim 15 wherein the desiccant is selected from a group consisting of silica, activated charcoal, calcium sulfate, calcium chloride and zeoliltes.

18. The hygienic system of claim 16 wherein the water-soluble glove dispenser contains a moisture indicator.

19. The hygienic system of claim 17 wherein the moisture indicator is cobalt chloride.

20.
